(19) United States
(12) Patent Application Publication
(10) Pub. No.: US 2025/0029725 A1
(43) Pub. Date: Jan. 23, 2025

(54) INFORMATION PROCESSING APPARATUS, INFORMATION PROCESSING METHOD, AND INFORMATION PROCESSING PROGRAM

(71) Applicant: FUJIFILM CORPORATION, Tokyo (JP)

(72) Inventor: Yu HASEGAWA, Tokyo (JP)

(21) Appl. No.: 18/905,153

(22) Filed: Oct. 3, 2024

Related U.S. Application Data
(63) Continuation of application No. PCT/JP2023/014934, filed on Apr. 12, 2023.

(30) Foreign Application Priority Data
Apr. 12, 2022 (JP) 2022-065906

(51) Int. Cl. G16H 50/20; G06T 7/00; G06V 10/25; G06V 20/50; G06V 20/70; G16H 15/00

(52) U.S. Cl. CPC G16H 50/20; G06T 7/0012; G06V 10/25; G06V 20/50; G06V 20/70; G16H 15/00; G06T 2207/30004; G06V 2201/031

(57) ABSTRACT

An information processing apparatus including a processor, wherein the processor is configured to: acquire a character string including a description regarding a first region of interest; specify a second region of interest that is not described in the character string and that is related to the first region of interest; and notify a user to check a necessity of displaying an image in which the second region of interest is likely to be included.

### CROSS REFERENCE TO RELATED APPLICATIONS

This application is a continuation of International Application No. PCT/JP2023/014934, filed on Apr. 12, 2023, which claims priority from Japanese Patent Application No. 2022-065906, filed on Apr. 12, 2022. The entire disclosure of each of the above applications is incorporated herein by reference.

### BACKGROUND

### Technical Field

The present disclosure relates to an information processing apparatus, an information processing method, and an information processing program.

### Related Art

In the related art, image diagnosis is performed using medical images obtained by imaging apparatuses such as computed tomography (CT) apparatuses and magnetic resonance imaging (MRI) apparatuses. Further, medical images are analyzed via computer-aided detection/diagnosis (CAD) using a discriminator in which learning is performed by deep learning or the like, and regions of interest including structures, lesions, and the like included in the medical images are detected and/or diagnosed. The medical images and analysis results via CAD are transmitted to a terminal of a healthcare professional such as a radiologist who interprets the medical images. The healthcare professional such as a radiologist interprets the medical images by referring to the medical images and analysis results using his or her own terminal and creates an interpretation report.

In addition, various methods have been proposed to support the creation of interpretation reports in order to reduce the burden of the interpretation work. For example, JP2019-153250A discloses a technology for creating an interpretation report based on a keyword input by a radiologist and on an analysis result of a medical image. In the technology disclosed in JP2019-153250A, a sentence to be included in the interpretation report is created by using a recurrent neural network trained to generate a sentence from input characters.

In addition, for example, JP2017-021648A discloses a technology for receiving a selection of a sentence from an input user, searching a report database based on the selected sentence, and extracting a sentence following the selected sentence. Furthermore, for example, JP2016-038726A discloses a technology for analyzing an interpretation report being input and creating a candidate for correction information to be used for correcting the interpretation report.

In interpreting medical images, after interpreting a certain region of interest, interpretation may also be performed on another related region of interest. For example, in a case in which a lesion is found in a lung field through interpretation, it is checked whether or not there is any associated lesion in other parts such as a mediastinal lymph node and a liver. Therefore, there is a need for a technology that can support interpretation of another region of interest related to a region of interest that has already been interpreted.

### SUMMARY

The present disclosure provides an information processing apparatus, an information processing method, and an information processing program that can support creation of an interpretation report.

According to a first aspect of the present disclosure, there is provided an information processing apparatus comprising a processor, in which the processor is configured to: acquire a character string including a description regarding a first region of interest; specify a second region of interest that is not described in the character string and that is related to the first region of interest; and notify a user to check a necessity of displaying an image in which the second region of interest is likely to be included.

According to a second aspect of the present disclosure, in the first aspect, the processor may be configured to specify the second region of interest related to the first region of interest based on correlation data in which a degree of association with other regions of interest is predetermined for each type of region of interest.

According to a third aspect of the present disclosure, in the second aspect, the correlation data may be determined based on a co-occurrence degree indicating a probability that two different types of the regions of interest appear simultaneously in a character string describing an image.

According to a fourth aspect of the present disclosure, in any one of the first to third aspects, the processor may be configured to display a character string indicating the second region of interest and at least one of a symbol or a figure on a display as the notification.

According to a fifth aspect of the present disclosure, in any one of the first to fourth aspects, the processor may be configured to display the image in which the second region of interest is likely to be included on a display.

According to a sixth aspect of the present disclosure, in the fifth aspect, the processor may be configured to, in a case in which the image includes the second region of interest, highlight the second region of interest.

According to a seventh aspect of the present disclosure, in the fifth or sixth aspect, the processor may be configured to, in a case in which there is an instruction, display the image in which the second region of interest is likely to be included on the display.

According to an eighth aspect of the present disclosure, in any one of the first to seventh aspects, the processor may be configured to: generate a character string including a description regarding the second region of interest; and display the character string on a display.

According to a ninth aspect of the present disclosure, in the eighth aspect, the processor may be configured to: acquire the image in which the second region of interest is likely to be included; and generate the character string including the description regarding the second region of interest based on the acquired image.

According to a tenth aspect of the present disclosure, in the eighth or ninth aspect, the processor may be configured to: generate a plurality of candidates of the character string including the description regarding the second region of interest; display the plurality of candidates of the character string on the display; and receive a selection of at least one of the plurality of candidates of the character string.

According to an eleventh aspect of the present disclosure, in any one of the first to tenth aspects, the processor may be configured to, in a case in which a plurality of the second regions of interest related to the first region of interest are specified, provide the notification in an order according to a priority of the second regions of interest.

According to a twelfth aspect of the present disclosure, in the eleventh aspect, the priority of the second regions of interest may be determined according to a degree of association with the first region of interest.

According to a thirteenth aspect of the present disclosure, in the eleventh or twelfth aspect, the priority of the second regions of interest may be determined according to findings of the second regions of interest diagnosed based on the image.

According to a fourteenth aspect of the present disclosure, in any one of the first to thirteenth aspects, the processor may be configured to: specify a finding of the first region of interest described in the character string; and specify the second region of interest related to the finding of the first region of interest.

According to a fifteenth aspect of the present disclosure, in any one of the first to fourteenth aspects, the image may be a medical image, and the first region of interest and the second region of interest may be each at least one of a region of a structure that is likely to be included in the medical image or a region of an abnormal shadow that is likely to be included in the medical image.

According to a sixteenth aspect of the present disclosure, there is provided an information processing method comprising: acquiring a character string including a description regarding a first region of interest; specifying a second region of interest that is not described in the character string and that is related to the first region of interest; and notifying a user to check a necessity of displaying an image in which the second region of interest is likely to be included.

According to a seventeenth aspect of the present disclosure, there is provided an information processing program for causing a computer to execute a process comprising: acquiring a character string including a description regarding a first region of interest; specifying a second region of interest that is not described in the character string and that is related to the first region of interest; and notifying a user to check a necessity of displaying an image in which the second region of interest is likely to be included.

The information processing apparatus, the information processing method, and the information processing program according to the aspects of the present disclosure can support creation of an interpretation report.

### BRIEF DESCRIPTION OF THE DRAWINGS

FIG. 1 is a diagram showing an example of a schematic configuration of an information processing system.

FIG. 2 is a diagram showing an example of a medical image.

FIG. 3 is a diagram showing an example of a medical image.

FIG. 4 is a block diagram showing an example of a hardware configuration of an information processing apparatus.

FIG. 5 is a block diagram showing an example of a functional configuration of the information processing apparatus.

FIG. 6 is a diagram showing an example of a screen displayed on a display.

FIG. 7 is a diagram showing an example of a screen displayed on a display.

FIG. 8 is a diagram showing an example of a screen displayed on a display.

FIG. 9 is a diagram showing an example of a screen displayed on a display.

FIG. 10 is a flowchart showing an example of information processing.

FIG. 11 is a diagram showing an example of a screen displayed on a display.

### DETAILED DESCRIPTION

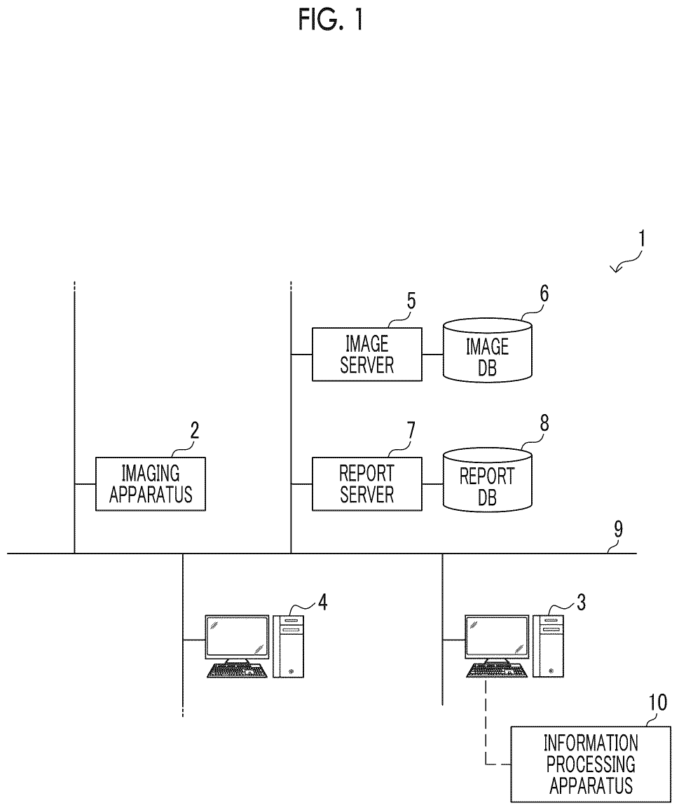

Hereinafter, embodiments of the present disclosure will be described with reference to the drawings. First, a configuration of an information processing system 1 to which an information processing apparatus of the present disclosure is applied will be described. FIG. 1 is a diagram showing a schematic configuration of the information processing system 1. The information processing system 1 shown in FIG. 1 performs imaging of an examination target part of a subject and storing of a medical image acquired by the imaging based on an examination order from a doctor in a medical department using a known ordering system. In addition, the information processing system 1 performs interpretation work of a medical image and creation of an interpretation report by a radiologist and viewing of the interpretation report by a doctor of a medical department that is a request source.

As shown in FIG. 1, the information processing system 1 includes an imaging apparatus 2, an interpretation workstation (WS) 3 that is an interpretation terminal, a medical care WS 4, an image server 5, an image database (DB) 6, a report server 7, and a report DB 8. The imaging apparatus 2, the interpretation WS 3, the medical care WS 4, the image server 5, the image DB 6, the report server 7, and the report DB 8 are connected to each other via a wired or wireless network 9 in a communicable state.

Each apparatus is a computer on which an application program for causing each apparatus to function as a component of the information processing system 1 is installed. The application program may be recorded on, for example, a recording medium, such as a digital versatile disc read-only memory (DVD-ROM) or a compact disc read-only memory (CD-ROM), and distributed, and be installed on the computer from the recording medium. In addition, the application program may be stored in, for example, a storage device of a server computer connected to the network 9 or in a network storage in a state in which it can be accessed from the outside, and be downloaded and installed on the computer in response to a request.

The imaging apparatus 2 is an apparatus (modality) that generates a medical image T showing a diagnosis target part of the subject by imaging the diagnosis target part. Examples of the imaging apparatus 2 include a simple X-ray imaging apparatus, a computed tomography (CT) apparatus, a magnetic resonance imaging (MRI) apparatus, a positron emission tomography (PET) apparatus, an ultrasound diagnostic apparatus, an endoscope, a fundus camera, and the like. The medical image generated by the imaging apparatus 2 is transmitted to the image server 5 and is stored in the image DB 6.

The interpretation WS 3 is a computer used by, for example, a healthcare professional such as a radiologist of a radiology department to interpret a medical image and to create an interpretation report, and encompasses an information processing apparatus 10 according to the present embodiment. In the interpretation WS 3, a viewing request for a medical image to the image server 5, various types of image processing for the medical image received from the image server 5, display of the medical image, and input reception of a sentence regarding the medical image are performed. In the interpretation WS 3, analysis processing for medical images, support for creating an interpretation report based on the analysis result, a registration request and a viewing request for the interpretation report to the report server 7, and display of the interpretation report received from the report server 7 are performed. The above processes are performed by the interpretation WS 3 executing software programs for respective processes.

The medical care WS 4 is a computer used by, for example, a healthcare professional such as a doctor in a medical department to observe a medical image in detail, view an interpretation report, create an electronic medical record, and the like, and is configured to include a processing device, a display device such as a display, and an input device such as a keyboard and a mouse. In the medical care WS 4, a viewing request for the medical image to the image server 5, display of the medical image received from the image server 5, a viewing request for the interpretation report to the report server 7, and display of the interpretation report received from the report server 7 are performed. The above processes are performed by the medical care WS 4 executing software programs for respective processes.

The image server 5 is a general-purpose computer on which a software program that provides a function of a database management system (DBMS) is installed. The image server 5 is connected to the image DB 6. The connection form between the image server 5 and the image DB 6 is not particularly limited, and may be a form connected by a data bus, or a form connected to each other via a network such as a network-attached storage (NAS) and a storage area network (SAN).

The image DB 6 is realized by, for example, a storage medium such as a hard disk drive (HDD), a solid-state drive (SSD), and a flash memory. In the image DB 6, the medical image acquired by the imaging apparatus 2 and accessory information attached to the medical image are registered in association with each other.

The accessory information may include, for example, identification information such as an image identification (ID) for identifying a medical image, a tomographic ID assigned to each tomographic image included in the medical image, a subject ID for identifying a subject, and an examination ID for identifying an examination. In addition, the accessory information may include, for example, information related to imaging such as an imaging method, an imaging condition, and an imaging date and time related to imaging of a medical image. The “imaging method” and “imaging condition” are, for example, a type of the imaging apparatus 2, an imaging part, an imaging protocol, an imaging sequence, an imaging method, the presence or absence of use of a contrast medium, a slice thickness in tomographic imaging, and the like. In addition, the accessory information may include information related to the subject such as the name, date of birth, age, and gender of the subject. In addition, the accessory information may include information regarding the imaging purpose of the medical image.

In a case in which the image server 5 receives a request to register a medical image from the imaging apparatus 2, the image server 5 prepares the medical image in a format for a database and registers the medical image in the image DB 6. In addition, in a case in which the viewing request from the interpretation WS 3 and the medical care WS 4 is received, the image server 5 searches for a medical image registered in the image DB 6 and transmits the found medical image to the interpretation WS 3 and to the medical care WS 4 that are viewing request sources.

The report server 7 is a general-purpose computer on which a software program that provides a function of a database management system is installed. The report server 7 is connected to the report DB 8. The connection form between the report server 7 and the report DB 8 is not particularly limited, and may be a form connected by a data bus or a form connected via a network such as a NAS and a SAN.

The report DB 8 is realized by, for example, a storage medium such as an HDD, an SSD, and a flash memory. In the report DB 8, an interpretation report created in the interpretation WS 3 is registered. Further, the report DB 8 may store finding information (details will be described later) regarding the medical image acquired in the interpretation WS 3.

Further, in a case in which the report server 7 receives a request to register the interpretation report from the interpretation WS 3, the report server 7 prepares the interpretation report in a format for a database and registers the interpretation report in the report DB 8. Further, in a case in which the report server 7 receives the viewing request for the interpretation report from the interpretation WS 3 and the medical care WS 4, the report server 7 searches for the interpretation report registered in the report DB 8, and transmits the found interpretation report to the interpretation WS 3 and to the medical care WS 4 that are viewing request sources.

The network 9 is, for example, a network such as a local area network (LAN) and a wide area network (WAN). The imaging apparatus 2, the interpretation WS 3, the medical care WS 4, the image server 5, the image DB 6, the report server 7, and the report DB 8 included in the information processing system 1 may be disposed in the same medical institution, or may be disposed in different medical institutions or the like. Further, the number of each apparatus of the imaging apparatus 2, the interpretation WS 3, the medical care WS 4, the image server 5, the image DB 6, the report server 7, and the report DB 8 is not limited to the number shown in FIG. 1, and each apparatus may be composed of a plurality of apparatuses having the same functions.

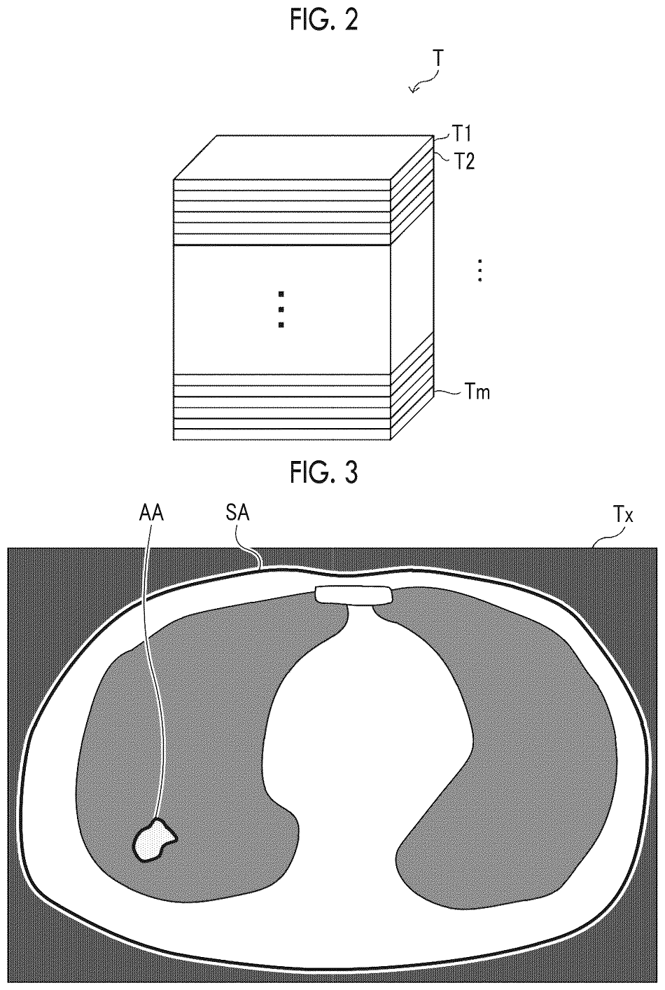

FIG. 2 is a diagram schematically showing an example of a medical image acquired by the imaging apparatus 2. A medical image T shown in FIG. 2 is, for example, a CT image consisting of a plurality of tomographic images T1 to Tm (m is 2 or more) representing tomographic planes from a head to a waist of one subject (human body). The medical image T is an example of an image of the present disclosure.

FIG. 3 is a diagram schematically showing an example of one tomographic image Tx out of the plurality of tomographic images T1 to Tm. The tomographic image Tx shown in FIG. 3 represents a tomographic plane including lungs. Each of the tomographic images TI to Tm may include a region SA of a structure showing various organs and viscera of the human body (for example, lungs, livers, and the like), various tissues constituting various organs and viscera (for example, blood vessels, nerves, muscles, and the like), and the like. In addition, each tomographic image may include a region AA of an abnormal shadow showing lesions such as, for example, nodules, tumors, injuries, defects, and inflammation. In the tomographic image Tx shown in FIG. 3, a lung region is the region SA of the structure, and a nodule region is the region AA of the abnormal shadow. A single tomographic image may include regions SA of a plurality of structures and/or regions AA of a plurality of abnormal shadows. Hereinafter, at least one of the region SA of the structure or the region AA of the abnormal shadow is referred to as a “region of interest”.

In interpreting medical images, after interpreting a certain region of interest, interpretation may also be performed on another related region of interest. For example, in a case in which a lesion is found in a lung field through interpretation, it is checked whether or not there is any associated lesion in other parts such as a mediastinal lymph node and a liver. Therefore, the information processing apparatus 10 according to the present embodiment has a function of supporting interpretation of another region of interest related to a region of interest that has already been interpreted (that is, a region of interest that has already been described in a comment on findings). The information processing apparatus 10 will be described below. As described above, the information processing apparatus 10 is encompassed in the interpretation WS 3.

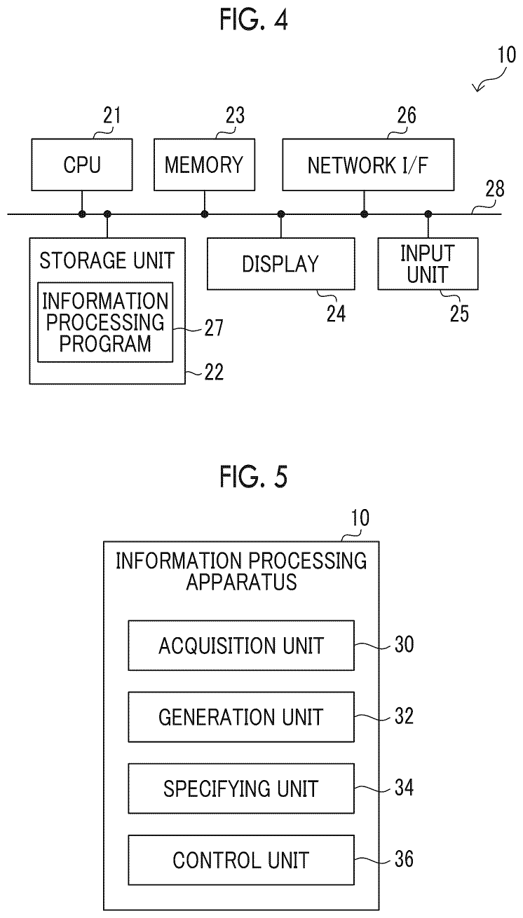

First, with reference to FIG. 4, an example of a hardware configuration of the information processing apparatus 10 according to the present embodiment will be described. As shown in FIG. 4, the information processing apparatus 10 includes a central processing unit (CPU) 21, a non-volatile storage unit 22, and a memory 23 as a temporary storage area. Further, the information processing apparatus 10 includes a display 24 such as a liquid-crystal display, an input unit 25 such as a keyboard and a mouse, and a network interface (I/F) 26. The network I/F 26 is connected to the network 9 and performs wired or wireless communication. The CPU 21, the storage unit 22, the memory 23, the display 24, the input unit 25, and the network I/F 26 are connected to each other via a bus 28 such as a system bus and a control bus so that various types of information can be exchanged.

The storage unit 22 is realized by, for example, a storage medium such as an HDD, an SSD, and a flash memory. An information processing program 27 in the information processing apparatus 10 is stored in the storage unit 22. The CPU 21 reads out the information processing program 27 from the storage unit 22, loads the read-out program into the memory 23, and executes the loaded information processing program 27. The CPU 21 is an example of a processor of the present disclosure. As the information processing apparatus 10, for example, a personal computer, a server computer, a smartphone, a tablet terminal, a wearable terminal, or the like can be applied as appropriate.

Next, with reference to FIGS. 5 to 9, an example of a functional configuration of the information processing apparatus 10 according to the present embodiment will be described. As shown in FIG. 5, the information processing apparatus 10 includes an acquisition unit 30, a generation unit 32, a specifying unit 34, and a control unit 36. In a case in which the CPU 21 executes the information processing program 27, the CPU 21 functions as each of the functional units of the acquisition unit 30, the generation unit 32, the specifying unit 34, and the control unit 36.

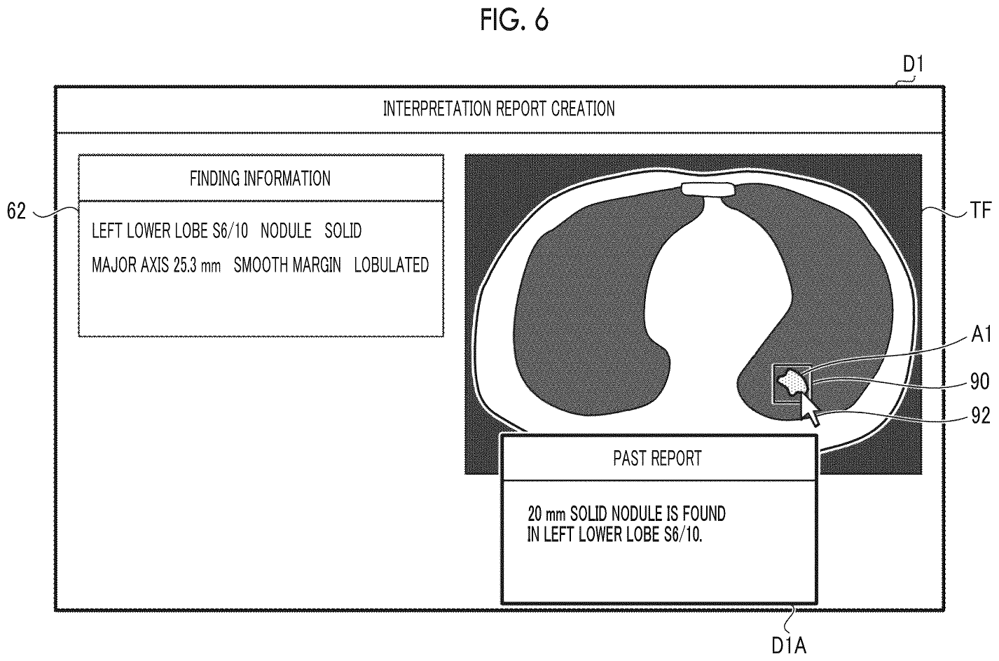

FIGS. 6 to 9 are diagrams showing examples of screens D1 to D4 displayed on the display 24 by the control unit 36, respectively. Hereinafter, functions of the acquisition unit 30, the generation unit 32, the specifying unit 34, and the control unit 36 will be described with reference to FIGS. 6 to 9.

### Interpretation of First Region of Interest

First, interpretation of a first region of interest A1 will be described with reference to the screen D1 in FIG. 6. The acquisition unit 30 acquires a medical image including the first region of interest A1 (hereinafter referred to as a “first image TF”) from the image server 5. On the screen D1, the first image TF is displayed under conditions suitable for interpretation of the lung field. The first region of interest A1 is at least one of a region of a structure that is likely to be included in the first image TF or a region of an abnormal shadow that is likely to be included in the first image TF.

In addition, the acquisition unit 30 acquires finding information about the first region of interest A1. As an example, the screen D1 shows finding information 62 in a case in which the first region of interest A1 is a nodule. The finding information includes, for example, information indicating various findings such as a name (type), a property, a position, a measurement value, and an estimated disease name.

Examples of names (types) include the names of structures such as “lung” and “liver”, and the names of abnormal shadows such as “nodule”. The property mainly means the features of abnormal shadows. For example, in the case of a lung nodule, findings indicating opacity such as “solid” and “ground-glass”, margin shapes such as “well-defined/ill-defined”, “smooth/irregular”, “spicula”, “lobulated”, and “lagged”, and an overall shape such as “round” and “irregular form” can be mentioned. Also, for example, the relationship with the peripheral tissue, such as “pleural contact” and “pleural invagination”, and findings regarding the presence or absence of contrast, washout, and the like can be mentioned.

The position means an anatomical position, a position in a medical image, and a relative positional relationship with other regions of interest such as “inside”, “margin”, and “periphery”. The anatomical position may be indicated by an organ name such as “lung” and “liver”, and may be expressed in terms of subdivided lungs such as “right lung”, “upper lobe”, and apical segment (“S1”). The measurement value is a value that can be quantitatively measured from a medical image, and is, for example, at least one of a size or a signal value of a region of interest. The size is represented by, for example, a major axis, a minor axis, an area, a volume, or the like of a region of interest. The signal value is represented by, for example, a pixel value in a region of interest, a CT value in units of HU, or the like. The estimated disease name is an evaluation result estimated based on the abnormal shadow. Examples of the estimated disease name include a disease name such as “cancer” and “inflammation” and an evaluation result such as “negative/positive”, “benign/malignant”, and “mild/severe” related to disease names and properties.

Specifically, the acquisition unit 30 may acquire finding information by extracting the first region of interest A1 from the acquired first image TF and performing image analysis on the first region of interest A1. As a method for extracting the first region of interest A1 from the first image TF, a known method using a CAD technology, an artificial intelligence (AI) technology, or the like can be appropriately applied. For example, the acquisition unit 30 may extract the first region of interest A1 from the first image TF by using a learning model such as a convolutional neural network (CNN) that has been trained to receive a medical image as an input and extract and output a region of interest included in the medical image. In addition, as a method of acquiring the finding information via image analysis, a known method using a CAD technology, an A1 technology, or the like can be appropriately applied. For example, the acquisition unit 30 may acquire finding information of the first region of interest A1 by using a learning model such as a CNN that has been trained in advance to receive the region of interest extracted from the medical image as an input and output the finding information of the region of interest.

In addition, the acquisition unit 30 inquires of the report server 7 as to whether or not an interpretation report created at a past point in time for the first region of interest A1 (hereinafter referred to as a “past report”) has been registered in the report DB 8. For example, in some cases, medical images are captured and interpreted a plurality of times for the same lesion of the same subject for follow-up observation. In this case, since the past report has already been registered in the report DB 8, the acquisition unit 30 acquires the past report from the report server 7. In this case, the acquisition unit 30 acquires a medical image including the first region of interest A1 captured at the past point in time (hereinafter referred to as a “past image”) from the image server 5.

As shown on the screen D1, the control unit 36 performs control to display the first image TF acquired by the acquisition unit 30 and its finding information 62 on the display 24. Furthermore, the control unit 36 may highlight the first region of interest A1 in the first image TF. For example, as shown on the screen D1, the control unit 36 may surround the first region of interest A1 with a bounding box 90 in the first image TF. For example, the control unit 36 may also add a marker such as an arrow near the first region of interest A1 in the first image TF, color-code the first region of interest A1 from other regions, or enlarge and display the first region of interest A1.

In addition, the control unit 36 may perform control to display past reports on the display 24 in a case in which a mouse pointer 92 operated by a user via the input unit 25 is placed over the first region of interest A1 on the screen D1 (so-called mouse hover/mouse over). In the example of FIG. 6, the mouse pointer 92 is placed over the first region of interest A1, and past reports for the first region of interest A1 are displayed on a pop-up screen D1A.

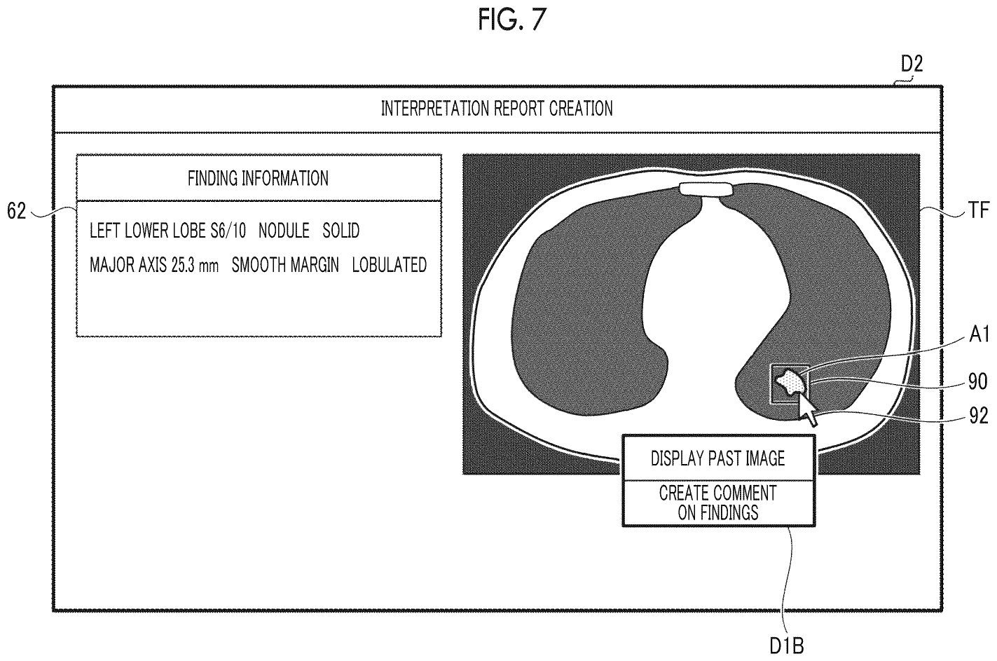

In addition, in a case in which the first region of interest A1 is selected by the mouse pointer 92 on the screen D1 (for example, in a case in which an operation such as clicking, double-clicking, or dragging is received), the control unit 36 may receive various operations regarding the first region of interest A1. FIG. 7 shows an example of a screen D2 that is transitioned to in the case in which the first region of interest A1 is selected on the screen D1. A menu D1B for receiving various operations regarding the first region of interest A1 is displayed on the screen D2. In a case in which “past image display” is selected in the menu D1B, the control unit 36 performs control to display the past image acquired by the acquisition unit 30 on the display 24 (not shown).

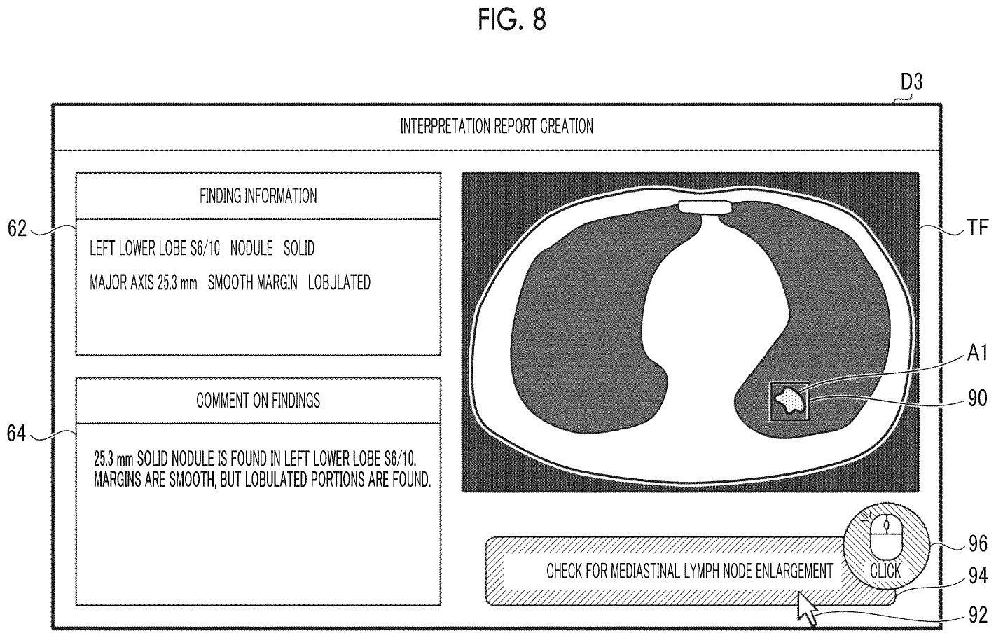

In a case in which “create comment on findings” is selected in the menu D1B, the generation unit 32 generates a comment on findings regarding the first region of interest A1. FIG. 8 shows an example of a screen D3 that is transitioned to in the case in which “create comment on findings” is selected in the menu D1B in FIG. 7. On the screen D3, a comment on findings 64 regarding the first region of interest A1 generated by the generation unit 32 is displayed.

Specifically, the generation unit 32 generates a comment on findings including the finding information 62 regarding the first region of interest A1 acquired by the acquisition unit 30. For example, the generation unit 32 may generate a comment on findings by using a method using machine learning such as the recurrent neural network described in JP2019-153250A. Further, for example, the generation unit 32 may generate a comment on findings by embedding the finding information 62 in a predetermined template. In addition, the generation unit 32 may receive corrections to the generated comment on findings from the user.

In a case in which the acquisition unit 30 inquires of the report server 7 and the past report is not registered in the report DB 8, the control unit 36 omits displaying the past report and the past image on the display 24.

### Checking of Necessity of Displaying Second Region of Interest

In a case in which the interpretation of the first region of interest A1 is completed in the manner described above, each functional unit checks with the user whether or not to also display another second region of interest A2 related to the first region of interest A1, that is, whether or not the user wishes to also interpret the second region of interest A2. The second region of interest A2 is at least one of a region of a structure that is likely to be included in the medical image or a region of an abnormal shadow included in the medical image. In addition, the medical image in which the second region of interest A2 is likely to be included may be an image obtained by imaging the same subject as the subject being imaged in the first image TF in which the first region of interest A1 is likely to be included, and may be the same image as the first image TF or a different image. In the following description, an example will be described in which the second region of interest A2 is included in a second image TS that is different from the first image TF.

Specifically, the acquisition unit 30 acquires a comment on findings including a description regarding the first region of interest A1 generated by the generation unit 32. The specifying unit 34 specifies a second region of interest A2 that is not described in the comment on findings acquired by the acquisition unit 30 and that is related to the first region of interest A1. For example, the specifying unit 34 specifies mediastinal lymph node enlargement as the second region of interest A2 that is not described in the comment on findings 64 in FIG. 8 and that is related to the first region of interest A1 (nodule) described in the comment on findings 64.

Specifically, the specifying unit 34 specifies a second region of interest A2 related to the first region of interest A1 based on correlation data in which a degree of association with other regions of interest is predetermined for each type of region of interest. For example, the correlation data may be determined based on a co-occurrence degree indicating the probability that two different types of regions of interest appear simultaneously in a character string (for example, a comment on findings) describing a medical image. For example, the specifying unit 34 may create correlation data indicating that the degree of association between “nodule” and “mediastinal lymph node enlargement” is relatively high in a case in which the number and/or percentage of comments on findings including “mediastinal lymph node enlargement” among the plurality of comments on findings including “nodule” registered in the report DB 8 is equal to or greater than a threshold value. The correlation data may be created in advance and stored in the storage unit 22 or the like, or may be created each time the second region of interest A2 is specified. In addition, the correlation data is not limited to the specifying unit 34 and may be created in an external device or the like.

Furthermore, for example, the correlation data may be determined based on guidelines, manuals, etc. that define the structures and/or lesions that should be checked simultaneously. In this case, the correlation data may be manually created by a user.

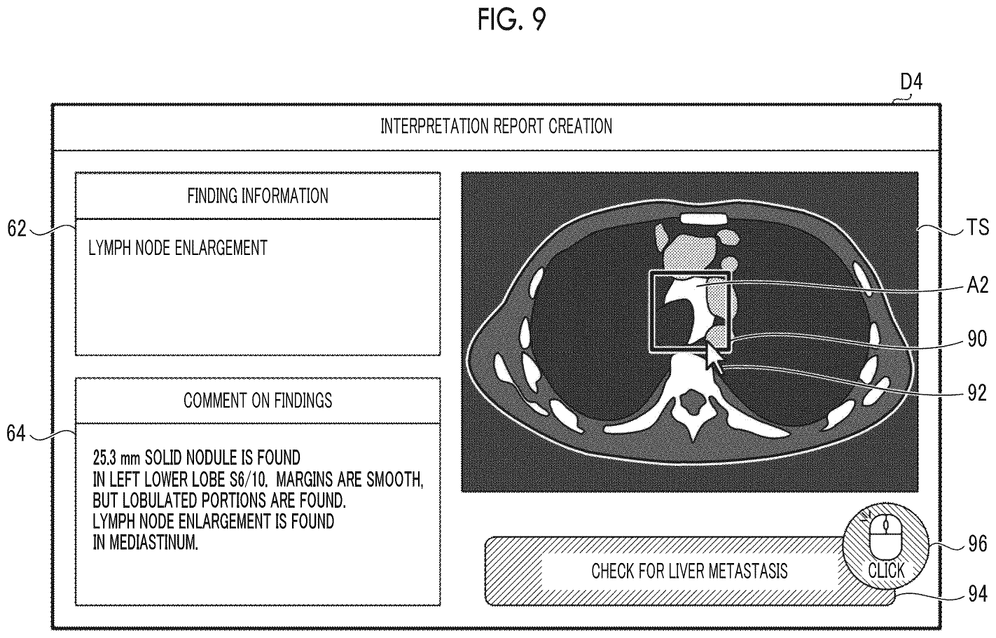

Furthermore, the specifying unit 34 specifies and acquires a second image TS in which the second region of interest A2 is likely to be included from among the medical images registered in the image server 5. For example, in a case in which the specifying unit 34 specifies mediastinal lymph node enlargement as the second region of interest A2, the specifying unit 34 specifies a medical image showing a tomographic plane including the mediastinal lymph node enlargement as the second image TS (see FIG. 9).

The second image TS may include the second region of interest A2, but does not necessarily have to include the second region of interest A2. For example, the finding of a nodule in the lung field does not necessarily lead to enlargement of the mediastinal lymph nodes. In this case, the specifying unit 34 may specify, as the second image TS, a medical image showing a tomographic plane including mediastinal lymph nodes in which no enlargement has occurred.

The control unit 36 notifies a user to check a necessity of displaying the second image TS in which the second region of interest A2 specified by the specifying unit 34 is likely to be included. This notification enables the user to recognize the presence of the second region of interest A2, and to decide whether or not to interpret the second image TS.

The screen D3 in FIG. 8 displays a notification 94 for checking whether or not the user checks the mediastinal lymph node enlargement specified as the second region of interest A2 (that is, whether or not the control unit 36 displays, on the display 24, a second image TS that may include mediastinal lymph node enlargement). An icon 96 for making the notification 94 more noticeable is also displayed on the screen D3. In this way, the control unit 36 may perform control to display a character string (notification 94) indicating the second region of interest A2 or at least one of a symbol or a figure (icon 96) on the display 24 as the notification. In addition, for example, the control unit 36 may provide a notification through sound output from a speaker and by means such as blinking of a light source like a light bulb or a light-emitting diode (LED).

### Interpretation of Second Region of Interest

After notifying the user as described above to check the necessity of displaying the second image TS, the control unit 36 may perform control to display, on the display 24, the second image TS in which the second region of interest A2 is likely to be included. Specifically, the control unit 36 may perform control to display the second image TS on the display 24 in a case in which there is an instruction from the user. For example, in a case in which the notification 94 is selected by the mouse pointer 92 on the screen D3 (for example, in a case in which an operation such as clicking or double-clicking is received), the control unit 36 may display the second image TS on the display 24. FIG. 9 shows an example of a screen D4 that is transitioned to in the case in which the notification 94 is selected on the screen D3 of FIG. 8. A second image TS is displayed on the screen D4.

Similarly to the interpretation of the first region of interest A1 described above, each functional unit may interpret the second region of interest A2. The functions of each functional unit related to the interpretation of the second region of interest A2 will be described below, but some of the functions similar to those for the interpretation of the first region of interest A1 will not be described.

The acquisition unit 30 acquires finding information about the second region of interest A2. Specifically, the acquisition unit 30 may acquire finding information by extracting the second region of interest A2 from the second image TS and performing image analysis on the second region of interest A2. As an example, the screen D4 shows finding information 62 in a case in which the second region of interest A2 is a lymph node enlargement.

In addition, the acquisition unit 30 inquires of the report server 7 as to whether or not an interpretation report created at a past point in time for the second region of interest A2 (hereinafter referred to as a “past report”) has been registered in the report DB 8, and acquires the interpretation report in a case in which it has already been registered. In this case, the acquisition unit 30 acquires a medical image including the second region of interest A2 captured at the past point in time from the image server 5.

As shown on the screen D4, the control unit 36 performs control to display the finding information 62 for the second region of interest A2 acquired by the acquisition unit 30 on the display 24. Furthermore, in a case in which the acquisition unit 30 analyzes that the second image TS includes the second region of interest A2, the control unit 36 may highlight the second region of interest A2 in the second image TS. For example, as shown on the screen D4, the control unit 36 may surround the second region of interest A2 with a bounding box 90 in the second image TS.

The control unit 36 may also display, on the display 24, an interpretation report that was created at a past point in time for the second region of interest A2 and that was acquired by the acquisition unit 30 (not shown). The control unit 36 may also perform control to display, on the display 24, a medical image including the second region of interest A2 captured at a past point in time, which was acquired by the acquisition unit 30 (not shown).

The generation unit 32 may generate a comment on findings including a description regarding the second region of interest A2. Specifically, the generation unit 32 may generate a comment on findings including finding information related to the second region of interest A2 acquired by the acquisition unit 30 based on the second image TS. That is, the generation unit 32 may generate a comment on findings including a description regarding the second region of interest A2 based on the acquired second image TS.

In addition, the control unit 36 performs control to display a comment on findings including a description regarding the second region of interest A2 generated by the generation unit 32 on the display 24. The screen D4 in FIG. 9 shows, as an example of a comment on findings generated by the generation unit 32, a comment on findings 64 in which a comment on findings regarding the second region of interest A2 (mediastinal lymph node enlargement) is added to a comment on findings regarding the first region of interest A1 (nodule) in FIG. 8.

### Interpretation of Plurality of Second Regions of Interest

The number of second regions of interest A2 related to the first region of interest A1 is not necessarily one. For example, in addition to the mediastinal lymph node enlargement, the checking of liver metastasis may be performed as another lesion related to the nodule of the lung field. Therefore, the specifying unit 34 may specify a plurality of second regions of interest A2 that are not described in the comment on findings acquired by the acquisition unit 30 and that are related to the first region of interest A1. In this case, each functional unit may perform interpretation of its respective second region of interest A2.

After completing the interpretation of the second image TS in which a certain second region of interest A2 is likely to be included, the control unit 36 may notify the user to check the necessity of displaying a medical image in which another second region of interest A2 is likely to be included. On the screen D4 of FIG. 9, a notification 94 is displayed for the user to check the necessity of displaying medical images in which liver metastasis is likely to be included on the screen D4 for interpreting mediastinal lymph node enlargement.

Furthermore, the control unit 36 may provide a notification in an order according to a priority of the plurality of second regions of interest A2 specified by the specifying unit 34. That is, the control unit 36 may notify the user to check the necessity of displaying medical images in which each second region of interest A2 is likely to be included, in an order according to the priority of each second region of interest A2. For example, it is assumed that mediastinal lymph node enlargement and liver metastasis are specified as the second region of interest A2 related to the nodule of the lung field, and the mediastinal lymph node enlargement is given a higher priority. In this case, the control unit 36 may notify the user to check the necessity of displaying medical images in which mediastinal lymph node enlargement is likely to be included prior to notifying the user to check the necessity of displaying medical images in which liver metastasis is likely to be included (that is, at the point in time at which interpretation of the nodule is completed).

The priority of each second region of interest A2 may be determined, for example, according to a degree of association with the first region of interest A1. The degree of association between the first region of interest A1 and the second region of interest A2 may be determined, for example, using correlation data in which a degree of association with other regions of interest is predetermined for each type of region of interest.

Furthermore, for example, the priority of each second region of interest A2 may be determined according to findings of the second regions of interest A2 diagnosed based on the medical image. For example, the control unit 36 may estimate the severity of the medical condition of each second region of interest A2 based on the finding information for each second region of interest A2 acquired by the acquisition unit 30, and may provide notifications in order of the severity of the medical condition.

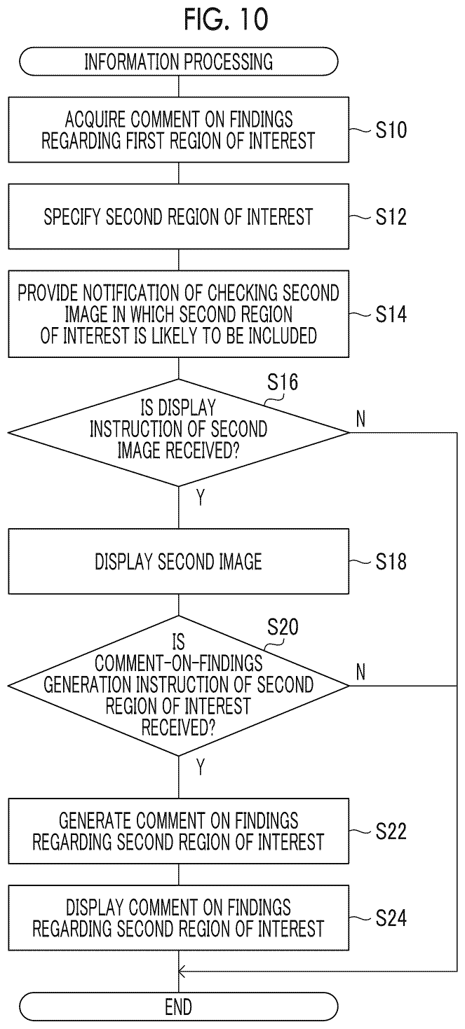

Next, with reference to FIG. 10, operations of the information processing apparatus 10 according to the present embodiment will be described. In the information processing apparatus 10, as the CPU 21 executes the information processing program 27, information processing shown in FIG. 10 is executed. The information processing is executed, for example, in a case in which the user provides an instruction to start execution via the input unit 25.

In Step S10, the acquisition unit 30 acquires a comment on findings including a description regarding the first region of interest A1. In Step S12, the specifying unit 34 specifies a second region of interest A2 that is not described in the comment on findings acquired in Step S10 and that is related to the first region of interest A1. In Step S14, the control unit 36 notifies a user to check a necessity of displaying the second image TS in which the second region of interest A2 specified in Step S12 is likely to be included.

In Step S16, the control unit 36 receives an instruction to display the second image TS on the display 24 (display instruction). That is, the user who has checked the notification in Step S14 inputs a display instruction of the second image TS as necessary. In a case in which the display instruction is received (Y in Step S16), the process proceeds to Step S18, and the control unit 36 performs control to display the second image TS on the display 24.

In Step S20, the control unit 36 receives an instruction to generate a comment on findings including the description regarding the second region of interest A2 (comment-on-findings generation instruction). That is, after checking the second image TS displayed on the display 24 in Step S18, the user inputs a comment-on-findings generation instruction regarding the second region of interest A2, as necessary. In a case in which the comment-on-findings generation instruction is received (Y in Step S20), the process proceeds to Step S22, and the generation unit 32 generates a comment on findings including a description regarding the second region of interest A2. In Step S24, the control unit 36 displays the comment on findings regarding the second region of interest A2 generated in Step S22 on the display 24 and ends the present information processing.

On the other hand, in a case in which the display instruction is not received (N in Step S16), the second image TS is not displayed and the present information processing ends. In addition, in a case in which the comment-on-findings generation instruction is not received (N in Step S20), the comment on findings is not generated and the present information processing ends.

As described above, the information processing apparatus 10 according to one aspect of the present disclosure comprises at least one processor. The processor acquires a character string including a description regarding a first region of interest, specifies a second region of interest that is not described in the character string and that is related to the first region of interest, and notifies a user to check a necessity of displaying an image in which the second region of interest is likely to be included.

That is, with the information processing apparatus 10 according to the present embodiment, the user can check the necessity of displaying the second image TS in which another second region of interest A2 related to the first region of interest A1 is likely to be included based on the comments on findings obtained by interpreting the first region of interest A1. Accordingly, it is possible to smoothly proceed with the interpretation of each of the first region of interest A1 that has been described in the comment on findings and the second region of interest A2 that has not been described in the comment on findings. Furthermore, the notification enables the user to recognize the presence of the second region of interest A2, thereby preventing the user from overlooking the second region of interest A2. Therefore, it is possible to support the creation of an interpretation report.

In addition, in the above embodiment, a form in which the acquisition unit 30 acquires finding information of the first region of interest A1 and the second region of interest A2 by performing image analysis on a medical image has been described, but the present disclosure is not limited thereto. For example, the acquisition unit 30 may acquire finding information stored in advance in the storage unit 22, the image server 5, the image DB 6, the report server 7, the report DB 8, and other external devices. Alternatively, for example, the acquisition unit 30 may acquire finding information manually input by the user via the input unit 25.

In addition, in the above embodiment, a form has been described in which the generation unit 32 generates one comment on findings including the description regarding the second region of interest A2 based on the second image TS, but the present disclosure is not limited thereto. For example, the generation unit 32 may acquire a comment on findings including a description regarding the second region of interest A2, which is stored in advance in the report DB 8, the storage unit 22, and other external devices, without depending on the second image TS. Furthermore, for example, the generation unit 32 may receive a comment on findings manually input by the user.

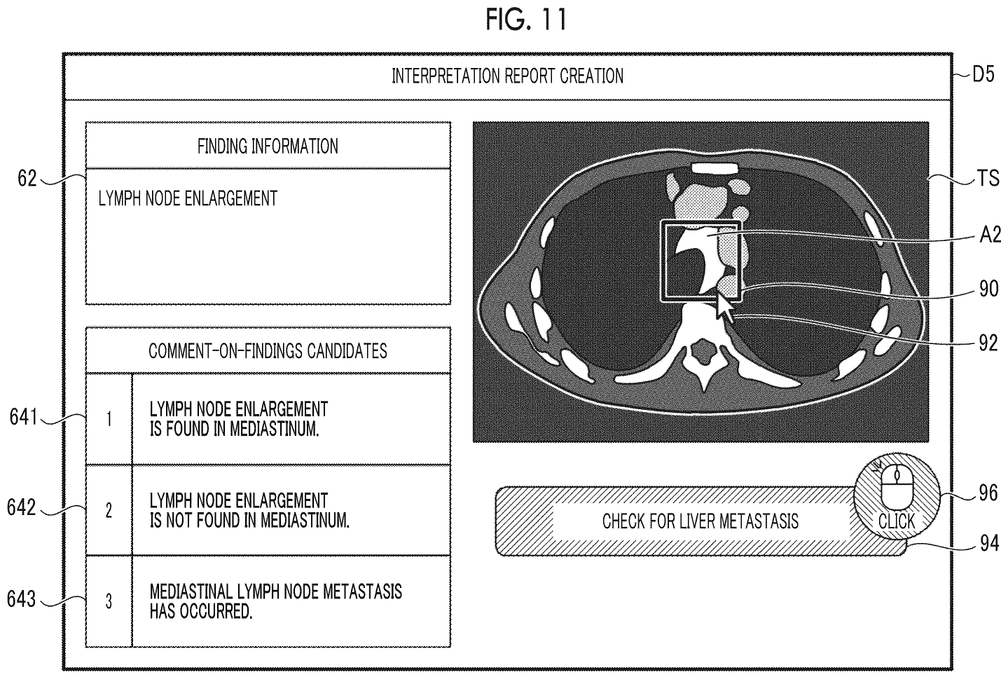

Further, for example, the generation unit 32 may generate a plurality of candidates of a comment on findings including a description regarding the second region of interest A2. FIG. 11 shows an example of a screen D5 on which a plurality of candidates 641 to 643 of a comment on findings regarding the second region of interest A2 (mediastinal lymph node enlargement) are displayed on the display 24 by the control unit 36. As shown in FIG. 11, the control unit 36 may display the plurality of candidates 641 to 643 of the comment on findings generated by the generation unit 32 on the display 24. In addition, the control unit 36 may receive a selection of at least one of the plurality of candidates 641 to 643 of the comment on findings.

In addition, in the above embodiment, a form of specifying the second region of interest A2 related to the first region of interest A1 has been described, but the present disclosure is not limited thereto. For example, even in the case of a nodule in the lung field, if the properties differ, such as “solid” and “ground glass”, the related second region of interest A2 may be different. Therefore, the specifying unit 34 may specify the findings of the first region of interest A1 described in the comment on findings including a description regarding the first region of interest A1 and may specify the second region of interest A2 related to the findings of the first region of interest A1.

Further, in the above embodiment, a form has been described in which various processes are performed using the comment on findings, but the present disclosure is not limited thereto. For example, instead of the comment on findings, various processes may be performed using documents such as interpretation reports, sentences including a plurality of comments on findings, and various character strings such as finding information and words included in the comments on findings.

In addition, in the above embodiment, a form has been described in which a nodule (that is, a region of an abnormal shadow) is used as an example of the first region of interest A1, and mediastinal lymph node enlargement (that is, a region of an abnormal shadow) is used as an example of the second region of interest A2, but the present disclosure is not limited thereto. As described above, the first region of interest A1 and the second region of interest A2 may each be at least one of a region of a structure that is likely to be included in the medical image or a region of an abnormal shadow that is likely to be included in the medical image, and the combination thereof is flexible.

For example, the first region of interest A1 may be a lung (that is, a region of a structure) and the second region of interest A2 may be a mediastinal lymph node (that is, a region of a structure). Further, for example, the first region of interest A1 may be a lung (that is, a region of a structure) and the second region of interest A2 may be mediastinal lymph node enlargement (that is, a region of an abnormal shadow). Further, for example, the first region of interest A1 may be a nodule (that is, a region of an abnormal shadow) and the second region of interest A2 may be a mediastinal lymph node (that is, a region of a structure).

Further, in the above embodiment, a form assuming an interpretation report for medical images has been described, but the present disclosure is not limited thereto. The information processing apparatus 10 according to one aspect of the present disclosure is applicable to various documents including descriptions regarding images obtained by imaging a subject. For example, the information processing apparatus 10 may be applied to documents including descriptions regarding images acquired using an apparatus, a building, a pipe, a welded portion, or the like as a subject in a non-destructive examination such as a radiation transmission examination and an ultrasonic flaw detection examination.

In addition, in the above embodiment, for example, as hardware structures of processing units that execute various kinds of processing, such as the acquisition unit 30, the generation unit 32, the specifying unit 34, and the control unit 36, various processors shown below can be used. As described above, the various processors include a programmable logic device (PLD) as a processor of which the circuit configuration can be changed after manufacture, such as a field-programmable gate array (FPGA), a dedicated electrical circuit as a processor having a dedicated circuit configuration for executing specific processing such as an application-specific integrated circuit (ASIC), and the like, in addition to the CPU as a general-purpose processor that functions as various processing units by executing software (programs).

One processing unit may be configured by one of the various processors, or may be configured by a combination of the same or different kinds of two or more processors (for example, a combination of a plurality of FPGAs or a combination of the CPU and the FPGA). In addition, a plurality of processing units may be configured by one processor.

As an example in which a plurality of processing units are configured by one processor, first, there is a form in which one processor is configured by a combination of one or more CPUs and software as typified by a computer, such as a client or a server, and this processor functions as a plurality of processing units. Second, as represented by a system-on-chip (SoC) or the like, there is a form of using a processor for realizing the function of the entire system including a plurality of processing units with one integrated circuit (IC) chip. In this way, various processing units are configured by one or more of the above-described various processors as hardware structures.

Furthermore, as the hardware structure of the various processors, more specifically, an electrical circuit (circuitry) in which circuit elements such as semiconductor elements are combined can be used.

In the above embodiment, the information processing program 27 is described as being stored (installed) in the storage unit 22 in advance; however, the present disclosure is not limited thereto. The information processing program 27 may be provided in a form recorded in a recording medium such as a compact disc read-only memory (CD-ROM), a digital versatile disc read-only memory (DVD-ROM), and a Universal Serial Bus (USB) memory. In addition, the information processing program 27 may be configured to be downloaded from an external device via a network. Further, the technology of the present disclosure extends to a storage medium for storing the information processing program non-transitorily in addition to the information processing program.

The technology of the present disclosure can be appropriately combined with the above embodiment and examples. The described contents and illustrated contents shown above are detailed descriptions of the parts related to the technology of the present disclosure, and are merely an example of the technology of the present disclosure. For example, the above description of the configuration, function, operation, and effect is an example of the configuration, function, operation, and effect of the parts related to the technology of the present disclosure. Therefore, it goes without saying that unnecessary parts may be deleted, new elements may be added, or replacements may be made to the described contents and illustrated contents shown above within a range that does not deviate from the gist of the technology of the present disclosure.

The disclosure of JP2022-065906 filed on Apr. 12, 2022 is incorporated herein by reference in its entirety. All documents, patent applications, and technical standards described in the present specification are incorporated in the present specification by reference to the same extent as in a case in which each of the documents, patent applications, and technical standards are specifically and individually indicated to be incorporated by reference.

What is claimed is:

1. An information processing apparatus comprising a processor, wherein the processor is configured to:
acquire a character string including a description regarding a first region of interest;
specify a second region of interest that is not described in the character string and that is related to the first region of interest; and
notify a user to check a necessity of displaying an image in which the second region of interest is likely to be included.

2. The information processing apparatus according to claim 1, wherein the processor is configured to specify the second region of interest related to the first region of interest based on correlation data in which a degree of association with other regions of interest is predetermined for each type of region of interest.

3. The information processing apparatus according to claim 2, wherein the correlation data is determined based on a co-occurrence degree indicating a probability that two different types of the regions of interest appear simultaneously in a character string describing an image.

4. The information processing apparatus according to claim 1, wherein the processor is configured to display a character string indicating the second region of interest and at least one of a symbol or a figure on a display as the notification.

5. The information processing apparatus according to claim 1, wherein the processor is configured to display the image in which the second region of interest is likely to be included on a display.

6. The information processing apparatus according to claim 5, wherein the processor is configured to, in a case in which the image includes the second region of interest, highlight the second region of interest.

7. The information processing apparatus according to claim 5, wherein the processor is configured to, in a case in which there is an instruction, display the image in which the second region of interest is likely to be included on the display.

8. The information processing apparatus according to claim 1, wherein the processor is configured to:
generate a character string including a description regarding the second region of interest; and
display the character string on a display.

9. The information processing apparatus according to claim 8, wherein the processor is configured to:
acquire the image in which the second region of interest is likely to be included; and
generate the character string including the description regarding the second region of interest based on the acquired image.

10. The information processing apparatus according to claim 8, wherein the processor is configured to:
generate a plurality of candidates of the character string including the description regarding the second region of interest;
display the plurality of candidates of the character string on the display; and
receive a selection of at least one of the plurality of candidates of the character string.

11. The information processing apparatus according to claim 1, wherein the processor is configured to, in a case in which a plurality of the second regions of interest related to the first region of interest are specified, provide the notification in an order according to a priority of the second regions of interest.

12. The information processing apparatus according to claim 11, wherein the priority of the second regions of interest is determined according to a degree of association with the first region of interest.

13. The information processing apparatus according to claim 11, wherein the priority of the second regions of interest is determined according to findings of the second regions of interest diagnosed based on the image.

14. The information processing apparatus according to claim 1, wherein the processor is configured to:
specify a finding of the first region of interest described in the character string; and
specify the second region of interest related to the finding of the first region of interest.

15. The information processing apparatus according to claim 1, wherein:
the image is a medical image, and
the first region of interest and the second region of interest are each at least one of a region of a structure that is likely to be included in the medical image or a region of an abnormal shadow that is likely to be included in the medical image.

16. An information processing method comprising:
acquiring a character string including a description regarding a first region of interest;
specifying a second region of interest that is not described in the character string and that is related to the first region of interest; and
notifying a user to check a necessity of displaying an image in which the second region of interest is likely to be included.

17. A non-transitory computer-readable storage medium storing an information processing program for causing a computer to execute a process comprising:
acquiring a character string including a description regarding a first region of interest;
specifying a second region of interest that is not described in the character string and that is related to the first region of interest; and
notifying a user to check a necessity of displaying an image in which the second region of interest is likely to be included.
